(19) United States
(12) Patent Application Publication
(10) Pub. No.: US 2023/0137369 A1
(43) Pub. Date: May 4, 2023

(54) AIDING A USER TO PERFORM A MEDICAL ULTRASOUND EXAMINATION

(71) Applicant: KONINKLIJKE PHILIPS N.V., EINDHOVEN (NL)

(72) Inventors: Marcin A Balicki, Cambridge, MA (US); Haibo Wang, Melrose, MA (US); Faik Can Meral, Mansfield, MA (US)

(21) Appl. No.: 17/918,146

(22) PCT Filed: Apr. 13, 2021
(86) PCT No.: PCT/EP2021/059470

§ 371 Date: Oct. 11, 2022

Related U.S. Application Data
(60) Provisional application No. 63/010,818, filed on Apr. 16, 2020.

(51) Int. Cl. A61B 8/00; A61B 8/08

(52) U.S. Cl. CPC A61B 8/461; A61B 8/5207; A61B 8/463; A61B 8/467

(57) ABSTRACT

A system for aiding a user to perform a medical ultrasound examination comprises a memory comprising instruction data representing a set of instructions; a processor; and a display. The processor is configured to communicate with the memory and to execute the set of instructions. The set of instructions, when executed by the processor, cause the processor to: i) receive a real-time sequence of ultrasound images captured by an ultrasound probe during the medical ultrasound examination; ii) use a model trained using a machine learning process to take an image frame in the real-time sequence of ultrasound images as input, and output a predicted relevance of one or more image components in the image frame to the medical ultrasound examination being performed; and iii) highlight to the user, in real-time on the display, image components that are predicted by the model to be relevant to the medical ultrasound examination, for further consideration by the user.

### TECHNICAL FIELD

The disclosure herein relates to ultrasound imaging. Particularly, but non-exclusively, embodiments herein relate to systems and methods for recording ultrasound images.

### BACKGROUND

Ultrasound (US) imaging is used in a range of medical applications such as, for example, fetal monitoring. Medical ultrasound imaging involves moving a probe comprising an ultrasound transducer that produces high frequency sound waves over the skin. The high frequency sound waves traverse through the tissue and reflect off internal surfaces (e.g. tissue boundaries). The reflected waves are detected and used to build up an image of the internal structures of interest.

Ultrasound imaging can be used to create two or three dimensional images. In a typical workflow, a user (e.g. sonographer, radiologist, clinician or other medical professional) may use two-dimensional imaging to locate an anatomical feature of interest. Once the feature is located in two dimensions, the user may activate a three-dimensional mode to take a three-dimensional image.

It is an object of embodiments herein to improve on such methods.

### SUMMARY

Sonographers are trained to perform ultrasound examinations to acquire image frames that capture normal features as well as those that contain pathologies. These images are later used by radiologists for diagnostics. The fact that image capture and image analysis may be performed by different people can lead to image views that are required by the radiologist not being captured by a sonographer. For example, inexperienced users (sonographers) may fail to capture sufficiently high quality images (in terms of depth, focus, number of views, etc.) with relevant diagnostic content either because they are inadequately trained, or don't pick up on the anatomical features and abnormalities that are considered most important by radiologists. This may lead to wasted time and resources, particularly if an ultrasound examination has to be repeated. It is an object of some embodiments herein to improve upon this situation.

Thus, according to a first aspect, there is a system for aiding a user to perform a medical ultrasound examination, the system comprises a memory comprising instruction data representing a set of instructions, a processor, and a display. The processor is configured to communicate with the memory and to execute the set of instructions. The set of instructions, when executed by the processor, cause the processor to: i) receive a real-time sequence of ultrasound images captured by an ultrasound probe during the medical ultrasound examination; ii) use a model trained using a machine learning process to take an image frame in the real-time sequence of ultrasound images as input, and output a predicted relevance of one or more image components in the image frame to the medical ultrasound examination being performed; and highlight to the user, in real-time on the display, image components that are predicted by the model to be relevant to the medical ultrasound examination, for further consideration by the user.

Thus according to this system, the user may be guided in real time to image the anatomical features that are predicted by a model trained using a machine learning process to be the most relevant to the medical ultrasound examination being performed. This helps ensure that the user does not miss relevant features that are salient to the diagnostic process.

According to a second aspect there is a method of aiding a user to perform a medical ultrasound examination. The method comprises receiving a real-time sequence of ultrasound images captured by an ultrasound probe during the medical ultrasound examination; using a model trained using a machine learning process to take an image frame in the real-time sequence of ultrasound images as input, and output a predicted relevance of one or more image components in the image frame to the medical ultrasound examination being performed; and highlighting to the user, in real-time on the display, image components that are predicted by the model to be relevant to the medical ultrasound examination, for further consideration by the user.

According to a third aspect there is a method of training a model for use in aiding a user to perform a medical ultrasound examination. The method comprises obtaining training data comprising: example ultrasound images; and ground truth annotations for each example ultrasound image, the ground truth annotations indicating a relevance of one or more image components in the respective example ultrasound image to the medical ultrasound examination; and training the model to predict a relevance to a medical ultrasound examination of one or more image components in an ultrasound image, based on the training data.

According to a fourth aspect there is a computer program product comprising computer readable medium, the computer readable medium having computer readable code embodied therein, the computer readable code being configured such that, on execution by a suitable computer or processor, the computer or processor is caused to perform the method of the second or third aspects.

### BRIEF DESCRIPTION OF THE DRAWINGS

For a better understanding and to show more clearly how embodiments herein may be carried into effect, reference will now be made, by way of example only, to the accompanying drawings, in which:

FIG. 1 shows an example system according to some embodiments herein;

FIG. 2 illustrates gaze tracking as used in some embodiments herein;

FIG. 3 illustrates an example method of training and using a neural network model according to some embodiments herein;

FIG. 4 illustrates an example neural network architecture according to some embodiments herein;

FIG. 5 illustrates an example system according to some embodiments herein;

FIG. 6 illustrates an example method according to some embodiments herein; and

FIG. 7 illustrates an example method according to some embodiments herein.

### DETAILED DESCRIPTION

As noted above, in general, there may be little feedback communication between radiologists and sonographers. Sonographers may follow standard imaging exam protocols in the hope that the standard is broad enough to cover the diagnostic imaging needs for every patient. Radiologists rarely directly influence the way the diagnostic imaging is performed for a particular patient.

In addition, quality assurance and sonographer performance assessment are generally limited, and mostly achieved through certification/training, and direct supervision.

Furthermore, the use of portable ultrasound is increasing, which may lead to less well-trained users performing ultrasound examinations in the field (e.g. emergency workers etc).

It is an aim of embodiments herein to provide intelligent real-time image interpretation and guidance aids for users of ultrasound imaging equipment in order to encourage imaging quality improvement and facilitate the use of ultrasound imagers by inexperienced users.

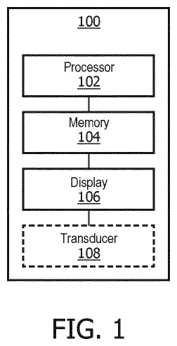

FIG. 1 illustrates a system (e.g. apparatus) 100 for recording ultrasound images according to some embodiments herein. The system 100 is for recording (e.g. acquiring or taking) ultrasound images. The system 100 may comprise or be part of a medical device such as an ultrasound system.

With reference to FIG. 1, the system 100 comprises a processor 102 that controls the operation of the system 100 and that can implement the method described herein. The processor 102 can comprise one or more processors, processing units, multi-core processors or modules that are configured or programmed to control the system 100 in the manner described herein. In particular implementations, the processor 102 can comprise a plurality of software and/or hardware modules that are each configured to perform, or are for performing, individual or multiple steps of the method described herein.

Briefly, the processor 102 of the system 100 is configured to: i) receive a real-time sequence of ultrasound images captured by an ultrasound probe during the medical ultrasound examination; ii) use a model trained using a machine learning process to take an image frame in the real-time sequence of ultrasound images as input, and output a predicted relevance of one or more image components in the image frame to the medical ultrasound examination being performed; and iii) highlight to the user, in real-time on the display, image components that are predicted by the model to be relevant to the medical ultrasound examination, for further consideration by the user.

In this way, the communication between sonographer and radiologist/physician may be minimized by capturing, in the model, the general body of knowledge from many radiologists of the features, regions or image components that they consider to be relevant features for a given image and given examination type. As will be described in more detail below, this information may be embedded, for example, in a large deep learning network and projected/highlighted onto the current US view to aid the sonographer in real time during the medical ultrasound examination. Technically, this may provide an improved manner in which to obtain ultrasound images to ensure that all views that are relevant (e.g. salient) to the medical ultrasound examination are adequately obtained. This may thus reduce the risk of an incorrect diagnosis being made, or an examination needing to be repeated due to inadequate data. The system may further be of use in remote imaging settings (e.g. imaging in the field, or at an emergency site) where there may not be a sonographer available to perform the medical examination. Generally, the system may be used to guide an untrained user to perform an ultrasound examination of acceptable quality.

In some embodiments, as illustrated in FIG. 1, the system 100 may also comprise a memory 104 configured to store program code that can be executed by the processor 102 to perform the method described herein. Alternatively or in addition, one or more memories 104 may be external to (e.g. separate to or remote from) the system 100. For example, one or more memories 104 may be part of another device. A memory 106 can be used to store images, information, data, signals and measurements acquired or made by the processor 102 of the system 100 or from any interfaces, memories or devices that are external to the system 100.

In some embodiments, as illustrated in FIG. 1, the system 100 may further comprise a transducer 108 for capturing ultrasound images. Alternatively or additionally, the system 100 may receive (e.g. via a wired or wireless connection) a data stream of two dimensional images taken using an ultrasound transducer 108 that is external to the system 100.

The transducer 108 may be formed from a plurality of transducer elements. Such transducer elements may be arranged to form an array of transducer elements. The transducer 108 may be comprised in a probe such as a handheld probe that can be held by a user (e.g. sonographer, radiologist or other clinician) and moved over a patient's skin. The skilled person will be familiar with the principles of ultrasound imaging, but in brief, ultrasound transducers comprise piezoelectric crystals that can be used both to generate and detect/receive sound waves. Ultrasound waves produced by the ultrasound transducer pass into the patient's body and reflect off the underlying tissue structures. Reflected waves (e.g. echoes) are detected by the transducer and compiled (processed) by a computer to produce an ultrasound image of the underlying anatomical structures, otherwise known as a sonogram.

In some embodiments the transducer 108 may comprise a matrix transducer that may interrogate a volume space.

In some embodiments, as illustrated in FIG. 1, the system 100 may also comprise at least one user interface such as a user display 106. The processor 102 may be configured to control the user display 106 to display or render, for example, the real-time sequence of ultrasound images captured by the ultrasound probe. The user display 106 may further be used to highlight to the user, in real-time, image components that are predicted by the model to be relevant to the medical ultrasound examination. This may be in the form of an overlay (e.g. markings, colorings or other shadings displayed over the real-time sequence of images, in a fully or partially transparent manner). The user display 106 may comprise a touch screen or an application (for example, on a tablet or smartphone), a display screen, a graphical user interface (GUI) or other visual rendering component.

Alternatively or in addition, at least one user display 106 may be external to (i.e. separate to or remote from) the system 100. For example, at least one user display 106 may be part of another device. In such embodiments, the processor 102 may be configured to send an instruction (e.g. via a wireless or wired connection) to the user display 106 that is external to the system 100 in order to trigger (e.g. cause or initiate) the external user displays to display the real-time sequence of ultrasound images and or highlight to the user, in real-time, image components that are predicted by the model to be relevant to the medical ultrasound examination.

It will be appreciated that FIG. 1 only shows the components required to illustrate this aspect of the disclosure, and in a practical implementation the system 100 may comprise additional components to those shown. For example, the system 100 may comprise a battery or other means for connecting the system 100 to a mains power supply. In some embodiments, as illustrated in FIG. 1, the system 100 may also comprise a communications interface (or circuitry) for enabling the system 100 to communicate with any interfaces, memories and devices that are internal or external to the system 100, for example over a wired or wireless network.

In more detail, the user may comprise the operator of the ultrasound probe, e.g. the person performing the ultrasound examination. Generally this may be a sonographer, a radiologist, or other medical practitioner. The user may be a person untrained in medical imaging, for example, a clinician or other user operating away from a medical setting, e.g. remotely or in the field. In such examples, the user may be guided by the system 100 in order to consider or image portions of the anatomy predicted to be important to a radiologist.

The medical ultrasound examination may comprise any type of ultrasound examination. For example, the model may be trained to determine relevant image components relevant to any type of (pre-specified) medical ultrasound examination. Examples of medical ultrasound examinations that the teachings herein may be applied to, include but are not limited to, an oncology examination for lesions, a neonatal examination of a fetus, an examination to assess a broken bone, or any other type of ultrasound examination.

As noted above, the set of instructions may cause the processor to, i) receive a real-time sequence of ultrasound images captured by an ultrasound probe during the medical ultrasound examination. In this sense, the processor may receive a sequence of ultrasound images from an ongoing ultrasound examination as the examination is in progress. As such, the sequence of ultrasound images may be considered a live stream or feed of ultrasound images, as captured by the ultrasound probe.

The sequence of ultrasound images may comprise a sequence of two-dimensional (2D), three-dimensional or any other dimensional ultrasound images. The ultrasound image frame may be comprised of image components. In a 2D image frame, the image components are pixels; in a 3D image frame the image components are voxels. The sequence of ultrasound images may be any type of ultrasound images, for example, B-mode images, Doppler ultrasound images, elastography mode images, or any other type or mode of ultrasound images.

In block ii) the processor is caused to use a model trained using a machine learning process to take an image frame in the real-time sequence of ultrasound images as input, and output a predicted relevance of one or more image components in the image frame to the medical ultrasound examination being performed.

The skilled person will be familiar with machine learning processes and models. However, in brief, the model may comprise any type of model that can be or has been trained, using a machine learning process to take an image (e.g. such as a medical image) as input and output a predicted relevance of one or more image components in the image frame to the medical ultrasound examination being performed. In some embodiments, the model may be trained according to the method 700 as described below.

In some embodiments, the model may comprise a trained neural network, such as a trained F-net or a trained U-net. The skilled person will be familiar with neural networks, but in brief, neural networks are a type of supervised machine learning model that can be trained to predict a desired output for given input data. Neural networks are trained using training data comprising example input data and the corresponding “correct” or ground truth outcome that is desired. Neural networks comprise a plurality of layers of neurons, each neuron representing a mathematical operation that is applied to the input data. The output of each layer in the neural network is fed into the next layer to produce an output. For each piece of training data, weights associated with the neurons are adjusted until the optimal weightings are found that produce predictions for the training examples that reflect the corresponding ground truths.

Although examples comprising neural networks are described herein, it will be appreciated that the teachings herein apply more generally to any type of model that can be used or trained to output a predicted relevance of one or more image components in the image frame to the medical ultrasound examination being performed. For example, in some embodiments, the model comprises a supervised machine learning model. In some embodiments, the model comprises a random forest model or a decision tree. The model may comprise a classification model, or a regression model. Examples of both types are provided in the text below. In other possible embodiments, the model may be trained using support-vector regression, or Random-Forest regression or other non-linear regressor. The skilled person will be familiar with these other types of supervised machine learning model that can be trained to predict a desired output for given input data.

In some embodiments, the trained model may have been trained using training data comprising: example ultrasound images; and ground truth annotations for each example ultrasound image, the ground truth annotations indicating a relevance of one or more image components in the respective example ultrasound image to the medical ultrasound examination. In this sense, the ground truth annotations represent an example of the “correct” prediction of which pixels in the example ultrasound image are relevant to the medical ultrasound examination.

The skilled person will be familiar with methods of training a machine learning model using training data. For example, gradient descent, back propagation, loss functions etc.

Generally, the training of the model may be performed incrementally by training the model onsite, e.g. at a site where radiologists review ultrasound images. Once trained, the trained model may then be installed on another system, e.g. an ultrasound machine. In other examples, the model may be located on a remote server and accessed and updated in a dynamic fashion. In other examples the model may be trained on historical data.

Briefly, the ground truth annotations may be obtained from one or more radiologists. In some examples, the ground truth annotations may be specific to the type of ultrasound examination being performed. For example, a model may be trained for a specific type of medical ultrasound examination. In such embodiments, the ground truth annotations may indicate image components or regions of the image relevant to that type of medical ultrasound examination. In other examples, the model may be trained for a more than one type of medical ultrasound examination. In such embodiments, the ground truth annotations may indicate image components or regions of the image that may be more generally relevant to many types of medical ultrasound examination.

In some embodiments, the annotations may comprise an image component level (e.g. pixel or voxel for 2D and 3D images respectively) annotation of the corresponding example ultrasound image frame, indicating a relevance, or relative relevance of each image component (e.g. pixel(s)/voxel(s)) in the image frame. In some embodiments this may be referred to as an annotation map, or annotation heat map.

The term “relevance” as used herein may be related to the level of importance that a radiologist would attribute to the image component or to different regions or groups of image components in the image frame, in the context of performing the medical ultrasound examination. For example, an image component or region of image components may be labelled as relevant if a radiologist would look at (e.g. consider or inspect) them as part of the ultrasound examination or wish to investigate them further.

In some embodiments, the ground truth annotations may be based on gaze tracking information obtained from observing a radiologist analyzing the respective example ultrasound image for the purpose of the medical ultrasound examination. Gaze tracking is a method to track the focal point of person's sight on a 2D screen. Gaze technology has improved significantly with data-driven models (see, for example the paper by Krafka et. al, 2016 entitled “Eye Tracking for Everyone”) and is precise whilst cost effective as it may be implemented using a simple camera and basic portable computing node. Using gaze tracking can enable collection and annotation of relevant input features without the user having to provide user input. E.g. the annotations may be collected as part of the normal examination of the images by the radiologist.

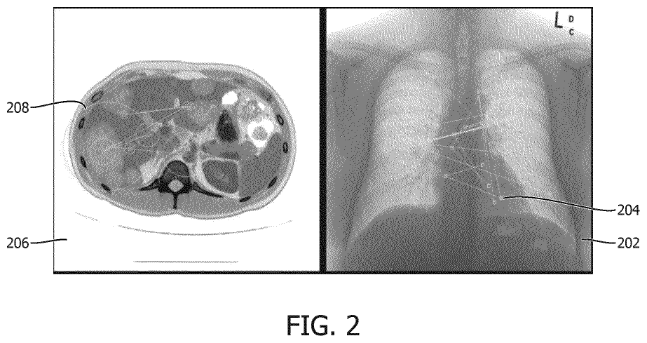

This is shown in FIG. 2 which illustrates different points that were observed by a radiologist in two ultrasound images of a chest cavity. In image 202, eye gaze data is represented as points 204 on the image that the radiologist looked at when analyzing the image. In the image 206, the gaze data is represented as a set of circular regions 208, centered on the points observed by the radiologist. The model may be trained, based on training data, to predict either type of annotation for a new (e.g. unseen) image. It will be appreciated that the model may also be trained to predict other outputs as described below.

Gaze information may be obtained by considering the location of the gaze and dwell time on the image while the radiologist/physician is inspecting the images. This “attention heat map” is then used as input where the dwell time is used as a relevancy score. In other words, a heat map may be produced from gaze information whereby the levels of the heat map are proportional to the amount of time the radiologist (or annotator) observed each particular region. A relevancy score may indicate, for example, a pathology or an area that is poorly visible, both of which are important to the sonographer for proper scanning.

In some embodiments, the model is trained to take as input an image frame from the real-time sequence of ultrasound images, e.g. just the image frame. In other embodiments the model may comprise additional input channels (e.g. take additional inputs). The model may take as input, for example, an indication of the type of medical ultrasound examination being performed. In other words a model may be trained to predict the relevance of pixels in an ultrasound image for different types of ultrasound examinations, depending on the indicated type of examination.

In some embodiments, the model may further be trained to take as input an indication of the likelihood of a user missing a feature. For example, the annotations may be graded according to both relevance and skill level of sonographer required before the user is likely to image the feature unprompted. This may enable the system to provide highlighting (e.g. in block iii) that is relevant to a user's experience level and/or to reduce the number of highlights provided by only providing relevant highlights that are most likely to be overlooked by a user.

Other examples of inputs may include radiologist annotations, sonographer's annotations, and ultrasound imaging settings, which may further improve the precision of the model. Other possible input parameters include an elastography or contrast image.

Turning now to the outputs of the model, in some embodiments, the output of the model (e.g. the predicted relevance of one or more image components in the image frame to the medical ultrasound examination being performed) may comprise a relevance value or score for each image component in the image frame. In such examples, the predicted relevance of the one or more image components in the image frame may comprise a map of the relevance values of each image component in the image frame.

In other examples, predicted relevance of one or more image components in the image frame may comprise relevance values or scores for a subset of image components in the image frame. For example, the subset of image components may have relevance values above a predetermined threshold relevance.

In some embodiments, regions of the ultrasound image frame may be pooled together using one or more relevance thresholds. In such embodiments, the predicted relevance of the one or more image components in the image frame may comprise one or more bounding boxes that encompasses image components, or regions of image components in the image frame with relevance values above a threshold (or between two thresholds). In some embodiments, the maximum relevance for each (or average) inside each bounding box may be provided as an output of the model.

The use of threshold in this manner may allow, for example, only the most relevant regions to be highlighted to the user, for example, only the top 10 percent of relevant image components. This may enable the sonographer to choose a particular threshold to show only the most pertinent annotations.

In some embodiments, the model may be trained to provide further outputs (e.g. have other output channels). For example, the model may be further trained to output an indication of a confidence associated with the predicted relevance for the one or more image components in the image frame.

In some examples, the confidence may comprise (or reflect) an estimated accuracy of the predicted relevance for the one or more image components as output by the model. Or a rating of how relevant the model determines each pixel/voxel to be in the image frame.

In other examples, the confidence for the one or more image components may comprise a prediction of a priority with which a radiologist would investigate a region comprising that image component, compared to other regions when performing the medical ultrasound examination. E.g. the confidence may comprise an estimation of the relative importance of different areas or different regions of image components (pixels/voxels) in the image frame.

In other examples, the output of the model may comprise a combination of the aforementioned options. For example, the confidence may comprise a measure of both the predicted relevance and the estimated accuracy of the prediction of the predicted relevance. In some embodiments, for each image component, the model may output (or the system may calculate from the outputs of the model) predicted relevance*estimated accuracy of the predicted relevance.

In block iii) the processor is then caused to highlight to the user, in real-time on the display, image components that are predicted by the model to be relevant to the medical ultrasound examination, for further consideration by the user.

For example, the processor may send an instruction to the display to cause the display to provide markings, annotations or overlays on the ultrasound frame to indicate the relevant areas of the image frame to the user. The user may thus be guided to give further consideration or to perform further imaging on areas of the anatomy that have been highlighted to the user.

In some embodiments, block iii) comprises the processor being caused to display the output of the model to the user in the form of a heatmap overlain over the ultrasound image frame. The levels of the heatmap may be based, for example, on the predicted relevance values for image components in the image frame. The levels of the heatmap may be colored or highlighted according to the relevance values. In this way the most relevant areas of the image frame may be effectively overlain with “bullseye” style annotations for the user to focus their imaging on.

In other embodiments, the levels of the heatmap may be based, for example, on the output confidences for image components in the image frame. The levels of the heatmap may be colored according to the confidence levels. In this way the most relevant areas of the image frame may be effectively overlain with “bullseye” style annotations for the user to focus their imaging on.

In other embodiments, the levels of a heatmap may be based on predicted relevance*estimated accuracy of the predicted relevance, as described above.

In embodiments where confidence is output, regions or areas of the image frame predicted as comprising image components of high relevance (e.g. above a threshold relevance) with high confidence (e.g. above a threshold confidence) may be annotated more prominently compared to other regions, as these comprise the areas that are most likely to be relevant to the medical ultrasound examination.

In other embodiments, where confidence is output, regions or areas of the image frame predicted as comprising image components of high relevance (e.g. above a threshold relevance) with low confidence (e.g. below a threshold confidence) may be annotated more prominently compared to other regions. In other words, it may be useful to show high salience areas that have low detection confidence, as these may represent, for example, a small lesion, or other feature that the radiographer may want to analyze in more detail. The sonographer can use this information to improve imaging quality of these regions.

In other embodiments, in block iii) processor may be caused to highlight to the user image components that are predicted by the model to be relevant to the medical ultrasound examination in any of the following ways (individually or in combination):
Bounding boxes, circles, or polygons with the color of the bounding box representing the relevance of image components in the region bounded
Bounding boxes, circles, or polygons with the scale/size representing relevance of image components in the bounded region
Bounding boxes, circles, or polygons with thickness representing the relevance of image components in the bounded region
Bounding boxes, circles, or polygons centered on the “center of gravity” of the image components in the region
Numerical value on or near the bounding box
Color each image component corresponding to the confidence in the relevance using a color map
Transparency (alpha blending of each image component with corresponding relevance as the weight and color mapping.
The highlighting may be dynamic in nature whereby relevant regions of image components within a certain proximity to a mouse cursor (e.g., within 2 cm circle) are highlighted to the user.

It will be appreciated that the processor may be further caused to repeat blocks ii) and iii) for a plurality of image frames in the real-time sequence of ultrasound images. For example the processor may be caused to repeat blocks ii and iii in a continuous (e.g. real-time) manner. The processor may be caused to repeat blocks ii and iii for all of the images in the real-time sequence of ultrasound images. Thus, in some embodiments, images from an ultrasound examination may be overlain with an annotation map as described above that changes in real time as the user moves the ultrasound probe. In this way, real-time guidance is provided to the user of the most relevant areas of the imaged anatomical features, to the medical ultrasound examination being performed.

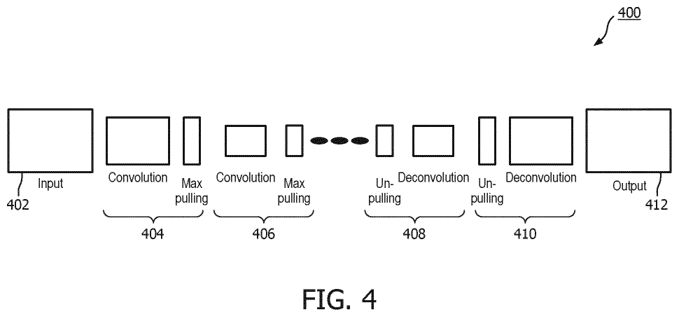

In some embodiments, a pixel-wise (or in 3D voxel-wise) flow model may be used to link the predicted relevance of image components in the image frame to a predicted relevance of image components in another image frame in the real-time sequence of ultrasound images. This may provide for a smooth overlay of highlighted relevant image components during the ultrasound examination. A pixelwise flow model may make use of temporal information of US imaging when available. In embodiments where the model outputs a map describing the relevance of each image component in the ultrasound image frame, the model may link the predicted maps of a subsequent ultrasound image frames in the sequence of ultrasound images, with regularization terms such as, for example, smoothness and temporal consistency. Relevance maps may be extended from pixelwise to voxelwise, predicting the salience level of each voxel in 3D space (see the paper by Girdhar et al. 2018, entitled “Detect-and-Track: Efficient Pose Estimation in Videos”: arXiv:1712.09184v2). The model architecture may be the same as the pixelwise relevance detection model (e.g. the same as that shown in FIG. 4 below).

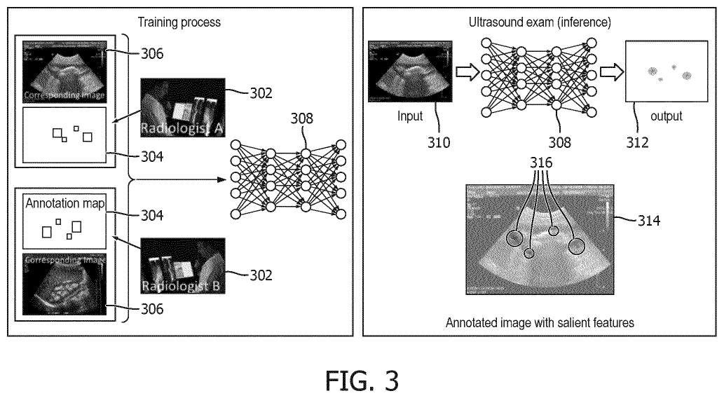

FIG. 3 shows a system according to some embodiments herein. In this embodiment, a model is trained to predict areas of an input ultrasound image that are relevant to a particular type of medical ultrasound examination. A plurality of radiologists 302 provide annotations of example ultrasound image frames 306 that are used as training data to train a neural network. In this embodiment, the annotations are in the form of annotation maps 304, comprising bounding boxes which indicate areas of each image frame that the annotating radiologist considers relevant to the type of medical examination being performed. The annotation maps are then used to train a neural network 308 to predict the ground truth annotation maps 304 from the input example ultrasound frames 306. In some versions of this embodiment, the neural network 308 may comprise the neural network 400 described below with respect to FIG. 4.

Once trained, the neural network 308 may be used in inference to take an image frame 310 in a real-time sequence of ultrasound images as input (e.g. an unseen image), and output a predicted annotation map 312 for that ultrasound image frame, indicating the relevance of each image component in the image frame to the medical ultrasound examination being performed. In this embodiment, the relevance is graded according to confidence as described above. As such, the annotation map has the appearance of a heat map, or plurality of “bullseye” style targets indicating the most relevant areas of the ultrasound frame.

A processor may then highlight to the user, in real-time on the display, image components that are predicted by the model to be relevant to the medical ultrasound examination, by overlaying the predicted annotation map over the ultrasound image 314 on the display to create heat map annotations (as highlighted by the white circles 316 in the image 314) over the image 314. This may be performed in real-time, e.g. such that the annotation map is overlain over the ultrasound image frame whilst the user is performing the ultrasound image. The user may thus be able to use the predicted annotation map as a guide to the regions of the image that should be considered, e.g. for further imaging.

Turning now to other embodiments, in one embodiment, as shown in FIG. 4, the model comprises a fully convolutional neural network, FCN 400. The FCN may be used to capture the salient features in ultrasound images defined manually in many ultrasound images by sonographers.

The FCN 400 takes an ultrasound frame as input (e.g. such as a 512×512 US image) via an input layer 402. The network first stacks one or more layers of convolution, batch normalization and pooling (max pooling in this graph) 404, 406. Each such layer can have a different number of convolutional kernels, strides, normalization operations and pooling kernel size. After each pooling, the input image will be down-sized proportionally to the size of the pooling kernel. On top of these layers, one or more unpooling and deconvolution layers 408, 410 are added to upsample the intermediate down-sized feature maps to the size of the original input image. In contrast to pooling, unpooling uses pixel interpolation to generate a larger image. The final output layer 412 outputs an originally-sized relevance map comprising relevance values or scores for each image component in the original image.

The whole architecture may be trained end-to-end using back propagation. The loss function in the last layer comprises a regression loss that is a sum of all the regression losses of regressing the feature maps of the last deconvolutional layer of each pixel to its labelled relevance (or salience) score.

Training data to train an FCN 400 as described above may be performed on diagnostic images and may involve direct annotation (e.g. via a mouse on a screen) by the annotating radiologist/physician. Any standard annotation tool is acceptable, such as a bounding box; center and circle radius, polygon, single click, etc. Furthermore, a saliency score (1-10, 10 being most important) may be given to each annotation region.

To reduce amount of data required for model training, the image set may be limited to a particular medical ultrasound examination (e.g. particular protocol or anatomical region being imaged). One way to do this is by adding an input into the network that specifies the type of medical ultrasound examination being performed, (or the step in the protocol being performed). In other embodiments, a separate deep learning model 400 may be trained for each step in a medical protocol.

In some embodiments, as illustrated in FIG. 4, the model may have the last fully connected layer being the same size as the input image size. In this way, the global per pixel context may be determined (e.g. a map may be built up on an image component basis, as was shown in FIG. 3). A transposed convolution layer (a.k.a., deconvolution) may enable this upsampling. The loss function in such embodiments may comprise per pixel regression. Standard data augmentation methods may be used to reduce bias in the data set, as well as to correct for sizing.

Exam Inference: Once the FCN as illustrated in FIG. 4 is trained, it can be used during routine ultrasound examinations (of the type that the FCN was trained for), where each image frame in a sequence of ultrasound images may be evaluated by (e.g. provided as input to) the trained model to output a corresponding annotation map where each pixel has a relevance score. As noted above, the relevance scores may then be used visually presented on the original US image used for the inference, as also illustrated in FIG. 3.

Turning now to other embodiments, in some examples, a patch-wise approach may be taken whereby the ultrasound frame is divided into smaller sub-frames. The subframes may be input into any of the embodiments of the model described herein. Once all the patches are processed, the outputs for the sub-frames may be combined to reconstruct the output for the full image frame (e.g. a map of the predicted relevance values of each image component in the image frame). For example, an annotation map may be reconstructed in the same configuration of the original image subdivisions. This may reduce the amount of data required for training because the relative location of the annotations is not considered. A similar alternative would use a bounding box region detector such as Yolo (see paper by Redmon et al. 2018 entitled: “YOLOv3: An Incremental Improvement”) that performs region detection at different scales.

In some embodiments, a classification model may be used. For example, in embodiments where the model outputs a relevance value or score, the score can be discretized . For example, a softmax layer in a neural network may be used to output the relevance values into predefined levels (e.g. such as 0.1, 0.2, 0.3, . . . , 1.0) which may be used as a classification label. This may reduce the computation power to implement the method described herein.

Whether an image component is relevant or not may be determined by the relative positions of different anatomical features in the image. For example, the relevance of annotations/features may be related to the presence of multiple features in a single view, and/or their spatial context (relative positions/orientations). Such information may be used in weighing the annotation importance, and may be embedded in training using a method such as, for example, Convolutional Pose Machines (see for example, the paper by Wei et al. entitled “Convolutional Pose Machines” 2016 IEEE Conference on Computer Vision and Pattern Recognition (CVPR), Las Vegas, Nev., 2016, pp. 4724-4732.) Put another way, in some embodiments, block ii) may comprises the processor being caused to take a relative spatial context of different anatomical features into account to predict the relevance of the one or more image components in the image frame. In some embodiments the processor may be caused to use a Convolutional Pose Machine trained to take the relative spatial context of different anatomical features into account to predict the relevance of the one or more image components in the image frame.

For quality assurance, it is possible to use gaze tracking during medical ultrasound examinations. For example, the system 100 may further comprise a camera and the processor may be further caused to monitor the gaze of the user with respect to the display 106 as they perform the ultrasound examination. In some embodiments, the user may be required to look at positions in the image frame that have been predicted by the model to be relevant (or above a particular relevance threshold). If it is determined from the gaze information that the user has not looked at an area of the ultrasound frame predicted by the model to be relevant, then the processor may be further configured to provide a visual aid to the user to prompt them to look at the relevant area. Put another way, the set of instructions, when executed by the processor, may further cause the processor to: determine gaze information of the user. In block iii) the processor may then be further caused to: highlight to the user, in real time on the display, one or more portions of the image frame that the gaze information indicates that the user has not yet looked at.

In some embodiments, the underlying image frame may need to be visible to the user. To make it easier for the user to see both the underlying image frame and the highlighting, in some embodiments, block iii) may further comprise the processor being caused to display markings highlighting the image components that are predicted by the model to be relevant to the medical ultrasound examination, and remove or fade the markings after a predetermined time interval. Put another way, temporarily visible markings that disappear after a few moments may be used.

In another example, in block iii) the processor may be caused to display markings highlighting the image components that are predicted by the model to be relevant to the medical ultrasound examination, wherein the markings are added or increased in prominence after a predetermined time interval. For example, the markings may be caused to become brighter over time if the user does not image (e.g. move the transducer towards) the region.

In another example, block iii may comprise the processor being configured to highlight components that are predicted by the model to be relevant to the medical ultrasound examination using a Head Up Display (HUD) or augmented reality.

In some embodiments, the system 100 may be used to train a user or sonographer. The system may, for example, be used without an ultrasound machine. The sonographer may be presented with a type of medical imaging procedure and one or more images from an examination database. For each image, the sonographer may be asked to select clinically important areas (using mouse or gaze as input) which may then be compared to the output of the model described herein.

The system 100 may be further configured to determine typical areas missed by users. This may be generic, or user specific. For example, over time a new (sonographer specific) model may be trained to highlight relevant image components that have been missed by the user. This may encode anatomical features in the model and displays them whenever they are detected. This is used to proactively guide the sonographer while reducing the clutter on the screen.

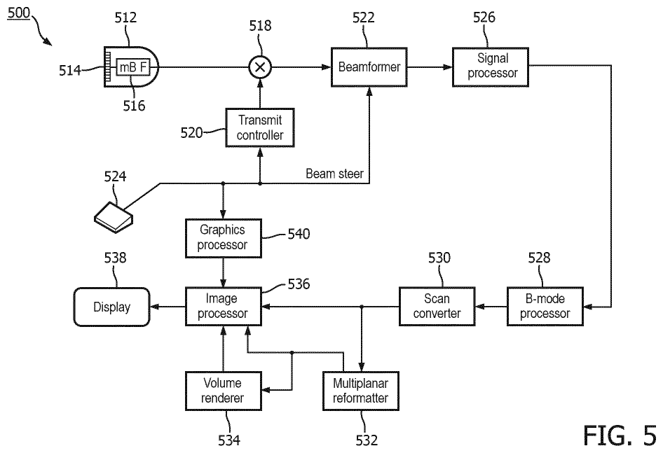

Turning now to FIG. 5, FIG. 5 shows an example embodiment of an ultrasound system 500, constructed according to the principles described herein. One or more components shown in FIG. 5 may be included within a system configured to i) receive a real-time sequence of ultrasound images captured by an ultrasound probe during the medical ultrasound examination; ii) use a model trained using a machine learning process to take an image frame in the real-time sequence of ultrasound images as input, and output a predicted relevance of one or more image components in the image frame to the medical ultrasound examination being performed; and iii) highlight to the user, in real-time on a display, image components that are predicted by the model to be relevant to the medical ultrasound examination, for further imaging consideration by the user.

For example, any of the above described functions of the processor 102 may be programmed, e.g., via computer executable instructions, into a processor of the system 500. In some examples, the functions of the processor 102 may be implemented and/or controlled by one or more of the processing components shown in FIG. 5, including for example, the image processor 536.

In the ultrasound imaging system of FIG. 5, ultrasound probe 512 includes a transducer array 514 for transmitting ultrasonic waves into a region of the body and receiving echo information responsive to the transmitted waves. The transducer array 514 may be a matrix array that includes a plurality of transducer elements configured to be individually activated. In other embodiments, the transducer array 514 may comprise a one-dimensional linear array. The transducer array 514 is coupled to a micro-beamformer 516 in the probe 512 which may control the transmission and reception of signals by the transducer elements in the array. In the example shown, the micro-beamformer 516 is coupled by the probe cable to a transmit/receive (T/R) switch 518, which switches between transmission and reception and protects the main beamformer 522 from high energy transmit signals. In some embodiments, the T/R switch 518 and other elements in the system can be included in the transducer probe rather than in a separate ultrasound system base.

The transmission of ultrasonic beams from the transducer array 514 under control of the microbeamformer 516 may be directed by the transmit controller 520 coupled to the T/R switch 518 and the beamformer 522, which receives input, e.g., from the user's operation of the user interface or control panel 524. One of the functions controlled by the transmit controller 520 is the direction in which beams are steered. Beams may be steered straight ahead from (orthogonal to) the transducer array, or at different angles for a wider field of view. The partially beamformed signals produced by the microbeamformer 516 are coupled to a main beamformer 522 where partially beamformed signals from individual patches of transducer elements are combined into a fully beamformed signal.

The beamformed signals are coupled to a signal processor 526. Signal processor 526 may process the received echo signals in various ways, such as bandpass filtering, decimation, I and Q component separation, and harmonic signal separation. Data generated by the different processing techniques employed by the signal processor 526 may be used by a data processor to identify internal structures, and parameters thereof.

The signal processor 526 may also perform additional signal enhancement such as speckle reduction, signal compounding, and noise elimination. The processed signals may be coupled to a B-mode processor 528, which can employ amplitude detection for the imaging of structures and tissues in the body. The signals produced by the B-mode processor are coupled to a scan converter 530 and a multiplanar reformatter 532. The scan converter 530 arranges the echo signals in the spatial relationship from which they were received in a desired image format. For instance, the scan converter 530 may arrange the echo signals into a two dimensional (2D) sector-shaped format. The multiplanar reformatter 532 can convert echoes which are received from points in a common plane in a volumetric region of the body into an ultrasonic image of that plane, as described in U.S. Pat. No. 6,663,896 (Detmer). A volume renderer 534 converts the echo signals of a 3D data set into a projected 3D image as viewed from a given reference point, e.g., as described in U.S. Pat. No. 6,530,885 (Entrekin et al).

The 2D or 3D images are coupled from the scan converter 530, multiplanar reformatter 532, and volume renderer 534 to an image processor 536 for further enhancement, buffering and temporary storage for display on an image display 538.

The graphics processor 540 can generate graphic overlays for display with the ultrasound images. These graphic overlays can contain, for example a map of relevance values or scores as output by the model described herein.

Graphic overlays may further contain other information, for example, e.g., standard identifying information such as patient name, date and time of the image, imaging parameters, and the like. The graphics processor may receive input from the user interface 524, such as a typed patient name. The user interface 524 may also receive input prompting adjustments in the settings and/or parameters used by the system 500. The user interface can also be coupled to the multiplanar reformatter 532 for selection and control of a display of multiple multiplanar reformatted (MPR) images.

The skilled person will appreciate that the embodiment shown in FIG. 5 is an example only and that the ultrasound system 500 may also comprise additional components to those shown in FIG. 5, for example, such as a power supply or battery.

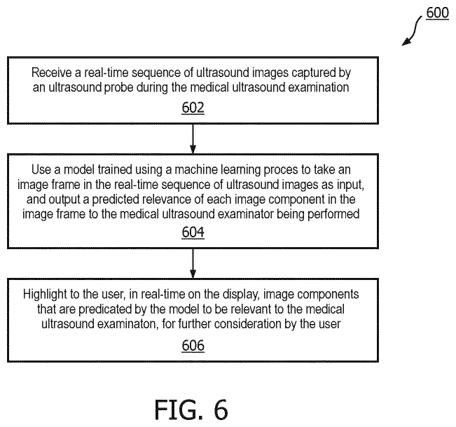

Turning now to FIG. 6, in some embodiments there is a method 600 of aiding a user to perform a medical ultrasound examination. The method may be performed, for example, by the system 100 or the system 700.

The method comprises in a block 602: receiving a real-time sequence of ultrasound images captured by an ultrasound probe during the medical ultrasound examination. In a block 604 the method comprises using a model trained using a machine learning process to take an image frame in the real-time sequence of ultrasound images as input, and output a predicted relevance of one or more image components in the image frame to the medical ultrasound examination being performed. In a block 606 the method comprises highlighting to the user, in real-time on the display, image components that are predicted by the model to be relevant to the medical ultrasound examination, for further consideration by the user.

Receiving a real-time sequence of ultrasound images captured by an ultrasound probe during the medical ultrasound examination was described in detail above with respect to the functionality of the system 100 and the detail therein will be understood to apply equally to block 602 of the method 600. Using a model trained using a machine learning process to take an image frame in the real-time sequence of ultrasound images as input, and output a predicted relevance of one or more image components in the image frame to the medical ultrasound examination being performed was described in detail above with respect to the functionality of the system 100 and the detail therein will be understood to apply equally to block 604 of the method 600. Highlighting to the user, in real-time on the display, image components that are predicted by the model to be relevant to the medical ultrasound examination, for further consideration by the user was described in detail above with respect to the functionality of the system 100 and the detail therein will be understood to apply equally to block 606 of the method 600.

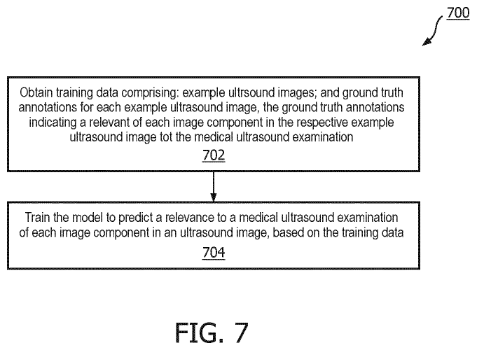

Turning to FIG. 7, in some embodiments, there is also a method 700 of training a model for use in aiding a user to perform a medical ultrasound examination. In a first block 702, the method 700 comprises obtaining training data comprising: example ultrasound images; and ground truth annotations for each example ultrasound image, the ground truth annotations indicating a relevance of one or more image component in the respective example ultrasound image to the medical ultrasound examination. In a second block 704, the method comprises training the model to predict a relevance to a medical ultrasound examination of image components in an ultrasound image, based on the training data. Training a model in this manner was discussed in detail above with respect to model described with respect to the system 100 and the detail therein will be understood to apply equally to the model 700.

In another embodiment, there is provided a computer program product comprising a computer readable medium, the computer readable medium having computer readable code embodied therein, the computer readable code being configured such that, on execution by a suitable computer or processor, the computer or processor is caused to perform the method or methods described herein.

Thus, it will be appreciated that the disclosure also applies to computer programs, particularly computer programs on or in a carrier, adapted to put embodiments into practice. The program may be in the form of a source code, an object code, a code intermediate source and an object code such as in a partially compiled form, or in any other form suitable for use in the implementation of the method according to the embodiments described herein.

It will also be appreciated that such a program may have many different architectural designs. For example, a program code implementing the functionality of the method or system may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system for aiding a user to perform a medical ultrasound examination, the system comprising:
a memory comprising instruction data representing a set of instructions;
a processor; and
a display;
wherein the processor is configured to communicate with the memory and to execute the set of instructions, and wherein the set of instructions, when executed by the processor, cause the processor to:
i) receive a real-time sequence of ultrasound images captured by an ultrasound probe during the medical ultrasound examination;
ii) use a model trained using a machine learning process to take an image frame in the real-time sequence of ultrasound images as input, and output a predicted relevance of one or more image components in the image frame to the medical ultrasound examination being performed; and
iii) highlight to the user, in real-time on the display, image components that are predicted by the model to be relevant to the medical ultrasound examination, for further consideration by the user.

2. A system as in claim 1 wherein the processor is further caused to repeat blocks ii) and iii) for a plurality of image frames in the real-time sequence of ultrasound images.

3. A system as in claim 1 wherein the model is trained using the machine learning process on training data comprising: example ultrasound images; and ground truth annotations for each example ultrasound image, the ground truth annotations indicating a relevance of one or more image components in the respective example ultrasound image to the medical ultrasound examination.

4. A system as in claim 3 wherein the ground truth annotations are based on gaze tracking information obtained from observing a radiologist analysing the respective example ultrasound image for the purpose of the medical ultrasound examination.

5. A system as in claim 1 wherein the model is further trained to output an indication of a confidence associated with the predicted relevance for the one or more image components in the image frame.

6. A system as in claim 5 wherein the confidence reflects an estimated accuracy of the predicted relevance for the one or more image components as output by the model.

7. A system as in claim 5 wherein the confidence for the one or more image components comprises a prediction of a priority with which a radiologist would investigate a region comprising that image component, compared to other regions when performing the medical ultrasound examination.

8. A system as in claim 5 wherein block iii) comprises the processor being caused to:
display the output of the model to the user in the form of a heatmap overlain over the ultrasound image frame, and wherein the levels of the heatmap are based on the output confidences for image components in the image frame.

9. A system as in claim 1 wherein block ii) comprises the processor being caused to take a relative spatial context of different anatomical features into account to predict the relevance of the one or more image components in the image frame.

10. A system as in claim 1 wherein the set of instructions, when executed by the processor, further cause the processor to:
determine gaze information of the user; and
wherein block iii) further comprises the processor being caused to:
highlight to the user, in real time on the display, one or more portions of the image frame that the gaze information indicates that the user has not yet looked at.

11. A system as in claim 1 wherein block iii) further comprises the processor being caused to:
display markings highlighting the image components that are predicted by the model to be relevant to the medical ultrasound examination, and wherein the markings are removed or faded after a predetermined time interval;
display markings highlighting the image components that are predicted by the model to be relevant to the medical ultrasound examination, and wherein the markings are added or increased in prominence after a predetermined time interval; and/or
highlight the image components that are predicted by the model to be relevant to the medical ultrasound examination using augmented reality.

12. A system as in claim 1 wherein the set of instructions, when executed by the processor, further cause the processor to:
use a pixel-wise flow model to link the predicted relevance of image components in the image frame to a predicted relevance of image components in another image frame in the real-time sequence of ultrasound images.

13. A method of aiding a user to perform a medical ultrasound examination the method comprising:
receiving a real-time sequence of ultrasound images captured by an ultrasound probe during the medical ultrasound examination;
using a model trained using a machine learning process to take an image frame in the real-time sequence of ultrasound images as input, and output a predicted relevance of one or more image components in the image frame to the medical ultrasound examination being performed; and
highlighting to the user, in real-time on the display, image components that are predicted by the model to be relevant to the medical ultrasound examination, for further consideration by the user.

14. A method of training a model for use in aiding a user to perform a medical ultrasound examination, the method comprising:
obtaining training data comprising: example ultrasound images; and ground truth annotations for each example ultrasound image, the ground truth annotations indicating a relevance of one or more image components in the respective example ultrasound image to the medical ultrasound examination; and
training the model to predict a relevance to a medical ultrasound examination of one or more image components in an ultrasound image, based on the training data.

15. A computer program product comprising computer readable medium comprising a computer readable medium, the computer readable medium having computer readable code embodied therein, the computer readable code being configured such that, on execution by a suitable computer or processor, the computer or processor is caused to perform the method as claimed in claim 13.
